(19) United States
(12) Patent Application Publication
(10) Pub. No.: US 2009/0233334 A1
(43) Pub. Date: Sep. 17, 2009

(54) CELL CULTIVATION AND PRODUCTION OF RECOMBINANT PROTEINS BY MEANS OF AN ORBITAL SHAKE BIOREACTOR SYSTEM WITH DISPOSABLE BAGS AT THE 1,500 LITER SCALE

(75) Inventors: Markus Hildinger, Pforzheim (DE); Florian Wurm, Monthey (CH); Matthieu Stettler, Monthey (CH); Maria DeJesus, Monthey (CH)

Correspondence Address: MARKUS HILDINGER, CRANACHWEG 8, PFORZHEIM, 75173, DE

(73) Assignee: EXCELLGENE SA, Monthey (CH)

(21) Appl. No.: 12/046,374

(22) Filed: Mar. 11, 2008

(51) Int. Cl. C12P 21/04; C12N 7/00; C12N 5/04; C12N 5/06; C12N 5/02; C12N 5/08; C12N 1/00

(52) U.S. Cl. 435/71.1; 435/235.1; 435/348; 435/358; 435/366; 435/394; 435/420; 435/243

(57) ABSTRACT

The present invention provides a novel method for culturing cells as well as a novel method for producing a recombinant protein by culturing cells at large scale (up to 1,500 L nominal volume and 750 L working volume), whereby an inflated bag provides a sterile, disposable cultivation chamber. The inflated bag is partially filled with liquid cultivation media and cells, and placed into a containment vessel. The containment vessel is positioned onto an orbitally shaken platform. The orbital shaking moves the containment vessel and thus the bag and induces thereby motion to the liquid contained therein (“shake mixing”). This motion (caused by orbital shaking) induces a dynamic force field that ensures cell suspension, bulk mixing, and oxygen transfer from the liquid surface to the respiring cells without damaging shear or foam generation.

### CROSS-REFERENCE TO RELATED APPLICATIONS

not applicable

### STATEMENT REGARDING FEDERALLY SPONSORED RESEARCH OR DEVELOPMENT

not applicable

### THE NAMES OF THE PARTIES TO A JOINT RESEARCH AGREEMENT

not applicable

### INCORPORATION-BY-REFERENCE OF MATERIAL SUBMITTED ON A COMPACT DISC

not applicable

### BACKGROUND OF THE INVENTION

It must be noted that as used herein and in the appended claims, the singular forms “a” and “the” include plural referents unless the context clearly dictates otherwise. Thus, for example, reference to “a cell” or “the cell” includes a plurality (“cells” or “the cells”), and so forth. Moreover, the word “or” can either be exclusive in nature (i.e., either A or B, but not A and B together), or inclusive in nature (A or B, including A alone, B alone, but also A and B together). One of skill in the art will realize which interpretation is the most appropriate unless it is detailed by reference in the text as “either A or B” (exclusive “or”) or “and/or” (inclusive “or”).

A portion of the disclosure of this patent document contains material which is subject to copyright protection. The copyright owner has no objection to the facsimile reproduction by anyone of the patent document or the patent disclosure as it appears in the Patent and Trademark Office file or records, but otherwise reserves all copyright rights whatsoever. The patent owners can be contacted at hildinger@gmx.net.

### FIELD OF THE INVENTION

The present invention relates to a novel cell culture apparatus (“bioreactor system”) useful for the cultivation of animal, insect, microbial, or plant cells in industrial or medical applications. More specifically, the present invention relates to a bioreactor system useful for the production of a recombinant protein by cultivation of animal, insect, microbial, or plant cells.

The present invention provides a novel cell culture apparatus (“bioreactor system”) for production of a recombinant protein by cultivation of animal, insect, microbial, or plant cells whereby an inflated bag provides a sterile, disposable cultivation chamber. The inflated bag is partially filled with liquid cultivation media and cells, and placed into a containment vessel. The containment vessel is positioned onto an orbital shaken platform. The orbital shaking moves the containment vessel and thus the bag and induces thereby motion to the liquid contained therein (“shake mixing”). This motion (caused by orbital shaking) induces a dynamic interface that ensures cell suspension, bulk mixing, and oxygen transfer from the liquid surface to the respiring cells without damaging shear or foam generation.

Culturing cells for the commercial production of proteins for diagnosis and therapy is a costly and time consuming process. The most commonly used equipment (stainless steel stirred bioreactors) is expensive (high investment cost), and production cost are high as well. Thus, methods and bioreactor systems are desirable which lower the upfront investment cost as well as the ongoing production cost. In order to achieve that, it is necessary to reduce the complexity of the bioreactor system by understanding and exploiting the unique characteristics of cell cultivation.

For such a bioreactor system to be successful, it must fulfill certain criteria:
(1) Eliminate gas bubbles which are known to cause cell damage.
(2) Eliminate high local shear caused by rotating mixers.
(3) Provide sufficient mixing to ensure a homogeneous environment, prevent cell settling, and promote gas transfer.
(4) Provide a sterile, disposable cultivation chamber to reduce labor cost and the need for steam sterilization.
(5) Reduce mechanical and instrumentation complexity to a minimum.
(6) Be scalable in order to allow large scale production of recombinant proteins with a nominal volume exceeding 1,000 liters and/or with a working volume exceeding 600 liters.
   The present invention will provide a new and improved method for culturing cells in vitro that achieves all these criteria, and overcomes all the aforementioned prior art limitations.

### DESCRIPTION OF RELATED ART INCLUDING INFORMATION DISCLOSED UNDER 37 CFR 1.97 AND 1.98

The present invention improves and combines existing technologies in the field of bioreactors and disposables used in the production of recombinant proteins by in vitro cell culture.

Bioreactors

Bioreactors play a key role in the field of biologics, where they are used for the production of recombinant therapeutic proteins by cultivation of animal cells. There are several types of bioreactors, including stirred-tank, orbital shake, Wave, airlift, hollow-fiber, and Rotary Cell Culture System (RCCS) designs. The conventional cell culture bioreactor for mammalian cells is a stirred tank that has been adapted from microbial cultivation by the addition of low-shear mixers and more gentle aeration systems (Armstrong et al U.S. Pat. No. 4,906,577 and Morrison U.S. Pat. No. 5,002,890).

Stainless steel stirred-tank bioreactors with sterilization in place (SIP)—the current “gold standard”—are expensive to acquire, install, maintain and operate. These bioreactors generally have a nominal volume of 100 L to 25,000 L and require elaborate mechanical systems to provide aeration and mixing. Control systems are required to sterilize the equipment and regulate temperature, pH and dissolved oxygen levels. Extensive training is required to operate these bioreactors without contamination. For these reasons, this type of bioreactor is primarily used in an industrial setting.

Whereas stainless steel stirred-tank bioreactors dominate in industrial applications, orbital shake bioreactors are the most frequently used reaction vessels in biotechnology and have been so for many decades. Their main area of usage has been for small scale (up to 10 liters) cultivation of primarily microbial cells. Only recently, their usage for the cultivation of eukaryotic cells has been explored (see the work of Liu et al.).

Disposables become increasingly important in biopharmaceutical manufacturing, and many companies are replacing rigid stainless steel and glass components with flexible, single-use plastics. Mixing tanks, filter assemblies, and tubing are some types of components that have successfully been replaced by disposable elements for cell culture production of high-value molecules.

Disposable bags are widely used in the biotechnology industry mainly for the purpose of sterile liquid handling. They are gamma sterilized and validated to match GMP requirements. More recently, and due to improvements made in material properties, disposable bags were designed for cell cultivation in Wave bioreactors or in stirred tank reactors with single-use contact parts. Such bags are equipped with sterile filters, connections and sampling ports. Normally, disposable bags are made with a polymeric film with at least three layers. The structural layer determines the overall mechanical behavior of the film. Then, a barrier layer defines the structure's permeability. Finally, the fluid contact layer combines inertness and good sealing properties. To match the regulatory requirements, the validation procedures for a new film consist in testing a variety of material properties including tensile properties, flex durability, permeability and possible interactions with the fluid. Further, to monitor the pH and the dissolved oxygen, innovative optical sensors can be integrated into the disposable cell culture bags. Sensor spots are immobilized on the inner layer of the bag in contact with the fluid. Using optical methods, the sensors can be assessed from the outside through the polymeric film. Optical sensors avoid contamination risks and can be discarded together with the cell culture bag. Response time and long term stability of optical sensors were improved to match process requirements.

Taking advantages of breakthroughs in disposable technologies, we have developed a reliable bioreactor system (based on orbital shaking principles) that allows high cell density cultures at nominal volumes exceeding 1,000 L and working volumes exceeding 600 L. This will establish single-use bioreactor technology as a new standard for cost effective and flexible recombinant protein production even at the production/large scale. The present invention—for the first time—describes how to cultivate cells and produce a recombinant protein with a bioreactor system based on orbital shaking and disposable bag technology at nominal volumes up to 1,500 L and working volumes up to 750 L.

Disposable Bioreactors

While single-use technologies are now widespread in many process steps, including filtration, sterile liquid handling, media and buffer preparation, the standard equipment for cell cultivation, e.g., the bioreactor itself, is predominantly non-disposable. Stirred tank and airlift bioreactors were initially developed for microbial production systems and were designed to achieve high gas transfer properties using direct gas dispersion into the liquid phase. They constitute well-defined and well-controlled environments that allow efficient process monitoring. For mammalian cells however, the design and the position of impellers and spargers were modified to reduce the hydrodynamic shear conditions, resulting in less efficient gas transfer properties. In recent years, systems have been developed that replace the conventional, commonly used stainless steel stirred bioreactors.

One commercially available option is the Wave bioreactor, a disposable bioreactor based on single-use bags. In 1998 Wave Biotech was the first company to commercialize a complete disposable cell cultivation system. The system included cell cultivation bags, filters, sampling system, aeration, agitation and monitoring, and is widely accepted and used for many applications at scales up to 500 L (working volume) and 1,000 L (nominal volume) and as a cell expansion system to feed stirred tank bioreactors. More recently, a number of other designs have entered the market, such as single-use stirred tank and air lift bioreactors based on disposable bag technology.

Though disposable bioreactors seem to be well accepted and used, major issues are still not solved. One key issue is the question of the largest possible operational scale. Unfortunately, current systems—including the Wave system—were not intended to reach production scales with 1,500 liter nominal volume and/or 750 liter working volume, but are limited to smaller scale applications. This is a drawback since the development and scale-up of a process currently relies on very different technologies when increasing the volumes from a few milliliters up to manufacturing scales. The present invention overcomes these limitations. Here, we present an alternative solution to the Wave bioreactor, which is based on orbital shaking and shake mixing, which allows successful cultivation of cells and production of recombinant proteins at a scale exceeding 600 liters of working volume and 1,000 liters of nominal volume.

Disposable Orbital Shake Bioreactors

Liu et al. were the first to scale-up production processes based on animal and insect cell lines in disposable shake bioreactors. They successfully cultivated hybridoma cells, Chinese hamster ovary (CHO) cells, and insect cell lines Sf-9 and H-5 (which demand higher oxygen rates than mammalian cells). They used cylindrical disposable reactors ranging from 3 to 50 liters. In all the cases, cell growth was better than that obtained by spinner flasks or a standard fermentor. Sf-9 cells were cultivated to a maximum viable cell density of >1×10⁷ cells/mL in 4 L and 20 L bioreactors. Similarly, H-5 cells were grown successfully in a 20 L shaking bioreactor to a viable cell density of 5×10⁶ cells/mL for scale-up production of recombinant proteins using a baculovirus/H-5 cell expression system.

Liu et al. also evaluated IgG production using hybridoma cells in shaking bioreactors from 3 L to 50 L. The experiments were conducted with 11% exchange/day of culture broth with fresh medium. IgG production reached 150 mg/L per day while maintaining 2×10⁶ viable cells/mL. A maximum of 250 mg/L of IgG was produced in the same process after termination of the daily exchange of broth and medium.

In addition, CHO cells were grown in a fed-batch mode in a 50 L shake bioreactor with a maximum viable cell count of 6×10⁶ cells/mL. Liu's group routinely uses 20 L scale shaking bioreactors with working volumes of 5-10 L to grow suspension-adapted mammalian (e.g., CHO, HEK293, hybridoma) and insect cells for recombinant protein expression and live cell production to support high throughput drug screening programs.

### BRIEF SUMMARY OF THE INVENTION

### (1) Substance or General Idea of the Claimed Invention

The present invention has been developed through many investigations to result in a low cost, simple solution to the problem of large scale cell culture (in the preferred embodiment: 1,500 liter nominal volume, 750 liter working volume). The bioreactor system of the present invention comprises a pre-sterilized flexible plastic bag in which cells are cultivated. The bag is partially filled with growth media and the remainder of the bag can be continuously purged with air or other oxygen-rich gas. The bag is placed into a containment vessel. Said containment vessel is placed on a platform that can be orbitally shaken. The orbital shaking motion promotes liquid movement in the bag which provides liquid mixing and enhances oxygen transfer from the headspace gas to the liquid phase where it is essential for cell growth and metabolism.

The present invention introduces a production scale shaker to hold a containment vessel with bags up to 1,500 liters nominal volume. The shaker is equipped with Kühner's direct drive technology, meaning that the speed of the motor is identical to the speed of the containment vessel. The advantage here is little noise, low power consumption due to less mechanical friction, and maintenance free operation. A parallelogram mechanism ensures that the shaking movement on the platform is absolutely equal and orbital, independent of the load distribution.

By using a disposable bag as the only contact surface for the cells, the bioreactor system provides excellent containment and eliminates labor intensive cleaning and sterilization. Lack of any mechanical parts except for the orbital shaker dramatically reduces cost and maintenance.

In a first aspect, the present invention provides a method for the cultivation of cells in a bioreactor system comprising the not necessarily consecutive steps of: (1) providing a bag having a nominal volume of at least 200 liters; (2) placing said bag into a containment vessel; (3) securing the containment vessel with the bag onto a platform of an orbital shaker; (4) introducing a liquid media and cells into the bag, wherein the liquid media and the cells comprise between 10% to 80% of the nominal volume and thus define the working volume; (5) filling the remainder of the bag with a gas; (6) orbitally shaking the platform to thereby induce motion to the liquid media in the bag, whereby the necessary oxygen transfer and mixing required for cell growth and/or survival is accomplished by the motion of the shake mixing.

In another aspect, the present invention provides a method for the production of a recombinant protein in a bioreactor system comprising the not necessarily consecutive steps of: (1) providing a bag having a nominal volume of at least 200 liters; (2) placing said bag into a containment vessel; (3) securing the containment vessel with the bag onto a platform of an orbital shaker; (4) introducing a liquid media and cells into the bag, wherein the liquid media and the cells comprise between 10% to 80% of the nominal volume and thus define the working volume; (5) filling the remainder of the bag with a gas; (6) orbital shaking the platform to thereby induce motion to the liquid media in the bag, whereby the necessary oxygen transfer and mixing required for cell survival and/or productivity is accomplished by the motion of the shake mixing.

In some embodiments, liquid media (with or without cells) is first introduced into the bag, and the bag is then placed into the containment vessel.

In some embodiments, the containment vessel with the bag is secured onto the platform after liquid is already inside the bag.

In some embodiments, step (5) (“filling the remainder of the bag with a gas”) is done by active aeration, i.e., by injection of air into the bag against a low back pressure. Yet, in other embodiments, step 5 is done passively, i.e., by passively letting air infuse inside the bag.

In a further aspect, the exact nominal volume of the bag should not be a limitation of the present invention as long as the nominal volume is at least 200 liters and preferentially more than 1,000 liters and most preferentially 1,500 liters. Thus, in some embodiments, the nominal volume of the bag can be 1,000 liters, in other embodiments it can be 1,500 liters. Similarly, the working volume of the bag can vary and will vary depending on a particular embodiment. Thus, in some embodiments, working volume can exceed 700 liters within a bag of a nominal volume of 1,500 liters. In the preferred embodiment, the working volume is 750 liters within a bag of a nominal volume of 1,500 liters.

In yet another aspect, gas can be introduced into the bag during the orbital shaking step. Such a gas can be oxygen and/or carbon dioxide. Similarly, in some embodiments, products of respiration are exhausted from the bag during the orbital shaking step.

In some embodiments, the bioreactor system of the present invention is used for biomass expansion; in some other embodiments, the bioreactor system is used for the production of a recombinant protein.

In some embodiments of the present invention the bag is disposable; in other embodiments of the present invention, the bag can also be reused. Furthermore, the exact material out of which the bag is made should not be a limitation of the present invention. The bag can be made out of plastic, and within the plastic category, out of different types of plastic such as polypropylene, polycarbonate or polyethylene, or any combination thereof. Similarly, the bag could have different shapes and geometries. It can be cylindrical, but also spherical. Thus, also the shape and geometry should not be perceived as a limitation of the present invention. In some embodiments, the bag can be inflated; in other embodiments, the bag cannot be inflated. Furthermore, the size (in terms of nominal volume) of the bag might vary from one embodiment to another embodiment and should not be considered a limitation of the present invention.

Similarly, depending on the exact embodiment, the shape, size and material of the containment vessel can vary as well. In some embodiments, a shape is placed at the bottom of the containment vessel. In the preferred embodiment, that shape is a metallic conical shape—resulting in a height difference of 180 mm.

In some embodiments, the bioreactor system of the present invention is non-instrumented.

In other embodiments, an optical sensor is used to measure the pH and dissolved oxygen within the bioreactor system.

In its preferred embodiment, the bioreactor system is equipped with the direct drive technology, which means that the speed of the motor is the same as the speed of the container.

In some embodiments, heating is provided by placing the bioreactor system in a heated environment such as a warm room. Yet, in some other embodiments, heating is provided by a heating system. One example of such a heating system is a silicone heating system, where large half-circle silicone heaters are adjusted to the containment vessel conical bottom and the cell culture bag is placed in direct contact with the heating elements.

In another aspect, the cells to be cultivated in the bioreactor system or the cells used to produce a recombinant protein within the bioreactor system can be of animal, human, insect, microbial or plant origin. In its preferred embodiment, cells are mammalian cells, and cells are used for the production of a recombinant protein such as a humanized monoclonal antibody. In particular, the cells are Chinese Hamster Ovary (CHO) cells.

This invention—for the first time—describes a method for cultivating cells in a disposable bioreactor system in general and a disposable bioreactor system based on orbital shaking in particular with nominal volumes exceeding 1,000 liters up to 1,500 liters. In another aspect, this invention—for the first time—describes a method for producing a therapeutically relevant protein in a disposable bioreactor system in general and a disposable bioreactor system based on orbital shaking in particular with nominal volumes exceeding 1,000 liters up to 1,500 liters.

Similarly, this invention—for the first time—describes a method for cultivating cells in a disposable bioreactor system in general and a disposable bioreactor system based on orbital shaking in particular with working volumes exceeding 580 liters up to 1,000 liters (750 liters in the preferred embodiment). In another aspect, this invention—for the first time—describes a method for producing a therapeutically relevant protein in a disposable bioreactor system in general and a disposable bioreactor system based on orbital shaking in particular with working volumes exceeding 580 liters up to 1,000 liters (750 liters in the preferred embodiment).

Furthermore, this invention—for the first time—describes a method for cultivating cells in a disposable bioreactor system in general and a disposable bioreactor system based on orbital shaking in particular with working volumes of 750 liters and a nominal volume of 1,500 liters (preferred embodiment). In another aspect, this invention—for the first time—describes a method for producing a therapeutically relevant protein in a disposable bioreactor system in general and a disposable bioreactor system based on orbital shaking in particular with working volumes of 750 liters and a nominal volume of 1,500 liters (preferred embodiment).

### (2) Advantages of the Invention Over Prior Approaches

### Usefulness of the Present Invention

The present invention is useful for animal, plant, microbial and insect cell culture, both in free suspension as well for anchorage-dependent systems. It is very suitable for virus and pathogen cultivation because of the high degree of containment.

Several advantages of the present invention apply to disposable bioreactors in general such as performance, flexibility, ease of handling, faster facility set-up, less maintenance and validation, reduced floor space and less capital investment. Yet, until recently, the major drawbacks of novel disposable shake bioreactors using single-use cell culture bags were the limitation in scale and problems in predicting the fluid dynamics at larger scales. The development of larger Wave type bioreactors was affected by such problems due to the complexity of the hydrodynamic behaviour and the almost endless number of combinations among bag geometry, filling volume, rocking speed, and rocking angle.

Other advantages of the present invention apply to the use of disposable materials in bioprocesses in general. The development of bioprocesses based on disposable materials is aimed at simplifying the technology for the production of biopharmaceuticals, resulting in several benefits. First, disposable systems increase the flexibility of bioprocesses. Compared to stainless steel equipment, the time required for changeovers between cell lines and batches is reduced, mainly because disposable systems require no cleaning and maintenance. Secondly, disposable systems reduce cost. In particular, the initial investment necessary for equipping a research and development lab or a pilot plant is less, and capital cost are exchanged by consumable cost, resulting in a more balanced cost distribution over time. Improved cost-effectiveness is particularly important in the context of competition and growing governmental and market price controls. In addition, accelerating the development process for a new therapeutic protein through increased flexibility and improved cost-effectiveness provides opportunities for achieving a competitive advantage.

A specific advantage of our orbital shake bioreactor system is that the gas-liquid interfacial area remains nearly constant during shaking and is well-defined in contrast to all other bioreactor types, especially Wave type bioreactors (FIG. 1). As a consequence, the scale-up of orbital shake systems is simpler as compared to the Wave bioreactor. Similar fluid dynamics are reproduced at different scales, resulting in more predictable oxygen transfer rates. Additionally, less foaming is expected as compared to stirred tank bioreactors and Wave systems.

The major advantage of the present invention is that—compared to the Wave bioreactor, our system is scalable beyond the scale of 600 liter working volume and 1,000 liter nominal volume. Scale is an important factor in biomanufacturing as it lowers cost. Whereas it is true that one can run 7×100 liter versus 1×700 liter, it is more cost effective to run one batch instead of seven (e.g., cost savings in terms of batch analytics; less risk of batch-to-batch variation).

To summarize: The key advantages of the present invention are that the present invention
Significantly reduces the cost of cell culture bioreactors compared to conventional glass and stainless steel stirred tank bioreactors. The low mechanical complexity of the present invention reduces operating and maintenance costs.
Provides a non-invasive means of agitation that reduces mechanical complexity and risk of contamination. This mode of agitation minimizes local shear that cause cell damage.
Improves cell growth and productivity by providing a bubble-free means of aeration that minimizes damage to cells caused by bubbles and foam formation. Prior art utilized mechanical mixers that impart high local shear, high-shear pump-around devices, or static culture that is incapable of scale-up.
Provides an easy to operate culture device suitable for industrial, laboratory and hospital environments. It eliminates the need for labor-intensive cleaning, preparation and sterilization, typical of conventional stainless steel bioreactor equipment by providing a pre-sterilized disposable one-use device.
Provides complete isolation of cells allowing cultivation in a non-aseptic environment, and is also useful for the culture of pathogens, viruses and other organisms requiring a high degree of containment.
Can be operated with widely varying culture volume. This allows for seed build-up within the culture vessel by adding media without the need for seed bioreactors and contamination-prone vessel-to-vessel transfers.
Leverages direct drive technology, meaning that the speed of the motor is identical to the speed of the containment vessel. The advantages here are little noise, low power consumption due to less mechanical friction, and maintenance free operation. A parallelogram mechanism ensures that the shaking movement on the platform is absolutely equal and orbital, independent of the load distribution.
Allows for large scale production—compared to other disposable systems such as the Wave system—with a nominal volume of 1,500 liters and a working volume of 750 liters in the preferred embodiment. To the best knowledge of the inventors, the present invention describes the largest scale bioreactor system based on disposable bag technology.

### Novelty of the Present Invention

As mentioned above, several bioreactor systems including disposable bioreactor systems and methods have to use them have been described in prior art. Yet, our disposable bioreactor system is novel in that it achieves large scale production at a scale not yet published in prior art. To the best knowledge of the inventors, the largest scale bioreactor system based on disposable bag technology in prior art is based on the Wave technology (www.wavebiotech.com) and is called SYSTEM500/1000 with a 500 liter working volume and a 1000 liter nominal volume, based on a CELLBAG1000L (with 100 to 500 liter working volume). Yet, the Wave company web site states “Units up to 580 liter culture have been operated successfully.”—making 580 liter working volume the highest scale published thus far. Our invention is novel and distinct in that the bioreactor system described exceeds 580 liters of working volume (750 liters of working volume in the preferred embodiment) and exceeds 1,000 liters of nominal volume (1,5000 liters of nominal volume in the preferred embodiment).

Furthermore, the bioreactor system of the present invention is not based on the Wave principle, but leverages orbital shaking. In that respect, the present invention is the first to demonstrate the achievement of 750 liters of working volume with 1,500 liters of nominal volume with disposable bag technology in the context of orbital shaking (see preferred embodiment).

In addition, in its preferred embodiment, direct drive technology is applied, meaning that the speed of the motor is identical to the speed of the containment vessel. This novelty has the advantage of little noise, low power consumption due to less mechanical friction, and maintenance free operation. A parallelogram mechanism ensures that the shaking movement on the platform is absolutely equal and orbital, independent of the load distribution.

This invention—for the first time—describes a method for cultivating cells in a disposable bioreactor system in general and a disposable bioreactor system based on orbital shaking in particular with nominal volumes exceeding 1,000 liters up to 1,500 liters. In another aspect, this invention—for the first time—describes a method for producing a therapeutically relevant protein in a disposable bioreactor system in general and a disposable bioreactor system based on orbital shaking in particular with nominal volumes exceeding 1,000 liters up to 1,500 liters.

Similarly, this invention—for the first time—describes a method for cultivating cells in a disposable bioreactor system in general and a disposable bioreactor system based on orbital shaking in particular with working volumes exceeding 580 liters up to 1,000 liters (750 liters in the preferred embodiment). In another aspect, this invention—for the first time—describes a method for producing a therapeutically relevant protein in a disposable bioreactor system in general and a disposable bioreactor system based on orbital shaking in particular with working volumes exceeding 580 liters up to 1,000 liters (750 liters in the preferred embodiment).

Furthermore, this invention—for the first time—describes a method for cultivating cells in a disposable bioreactor system in general and a disposable bioreactor system based on orbital shaking in particular with working volumes of 750 liters and a nominal volume of 1,500 liters (preferred embodiment). In another aspect, this invention—for the first time—describes a method for producing a therapeutically relevant protein in a disposable bioreactor system in general and a disposable bioreactor system based on orbital shaking in particular with working volumes of 750 liters and a nominal volume of 1,500 liters (preferred embodiment).

### Non-Obviousness of the Present Invention

The present invention combines multiple aspects in a non-obvious way to achieve a non-instrumented, disposable bioreactor for the cultivation of mammalian cells and the production of recombinant proteins at a working volume scale exceeding 580 liters. For instance, in its preferred embodiment, the present invention combines disposable plastic bags for media preparation with a self-made containment vessel and a prototype orbital shaker from Adolf Kühner AG (Switzerland; www.kuhner.com).

Whereas individual elements of the present invention are in the public domain, it is not obvious that the combination of those elements will yield a bioreactor system which allows for cultivation of cells and the production of recombinant proteins with working volumes exceeding 580 liters and nominal volumes exceeding 1,000 liters.

Given the high commercial interest in biomanufacturing at low cost and the long history of cultivating bacterial cells with orbital shaking at lower (less than 100 liters) working volumes, it should not have been obvious to one of ordinary skill in the art that working volumes can be successfully increased to volumes exceeding 580 liters by means of the hereby disclosed invention.

### BRIEF DESCRIPTION OF THE SEVERAL VIEWS OF THE DRAWING(S)

FIG. 1: Properties of the air-liquid mass transfer area in bioreactors for animal cell culture.

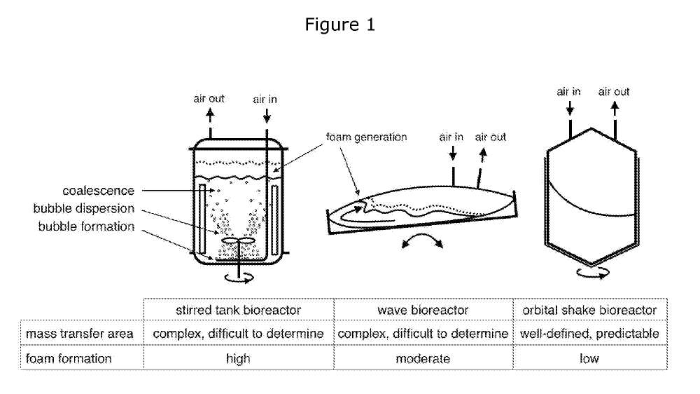

FIG. 2: (A) 200 L shake bioreactor and modified large-capacity shaker (left). The production scale bioreactor was inoculated with 75 L of cells expanded using the 200 L shake bioreactor (right). (B) 1,500 L disposable shake bioreactor with a cell culture volume of 750 L and agitated at 43 rpm. Height: 2 m, floor space: 4 m², weight empty: 1,000 kg.

FIG. 3: Schematic drawing of the cylindrical disposable cell culture bags. The 200 L HyClone bag was a standard item. The 1,500 L Lonza bag was designed and manufactured for this project. The ready-to-use bags were sterilized by gamma irradiation.

FIG. 4: Inside view of the container showing the conical geometry of the bottom and the silicone heat elements.

FIG. 5: Schematic description of the optical oxygen sensing set-up for the 200 L (left) and the 1,500 L (right) shake systems.

FIG. 6: Disposable shake bioreactors: Scale-up sequence from mL scale to production scale. 50 mL and 1 L shake bioreactors are passively aerated, whereas larger systems are actively aerated. The arrows represent the inlet and outlet airflows.

FIG. 7: Large-scale data. 75 L with CHO protein-free medium (Sigma). 750 L with proCHO5 media (Lonza).

FIG. 8: Summary of some key results and parameters of the preferred embodiment in an overview table.

### DETAILED DESCRIPTION OF THE INVENTION

### (1) Definitions

In describing the present invention, the following terms will be employed, and are intended to be defined as indicated below.

For purpose of this invention, the term “large scale” or “production scale”—as it relates to disposable bag technology—refers to a production process in general and a bag in particular with a nominal volume exceeding 1,000 liters and/or a working volume exceeding 600 liters.

For purpose of this invention, the term “large scale orbital shake bioreactor system” or “production scale orbital shake bioreactor system” or “large scale orbital shake bioreactor” or “production scale orbital shake bioreactor” refers to a bioreactor system capable of handling bags with more than 600 liter working volume and/or 1,000 liter nominal volume and comprises at least the following components: (1) An orbital shaker, (2) a containment vessel (for a disposable plastic bag), which can be mounted on the orbital shaker, and (3) a disposable plastic bag, which contains the cells in a liquid medium and can be placed inside the containment vessel. The system might further comprise two optional components, a heating system (4) and optical sensors (5). As for temperature control, the orbital shake bioreactor can be placed in a temperature-controlled environment, which is not part of the orbital shake bioreactor system (e.g., a 37° C. room), which eliminates the need for an endogenous heating system. Alternatively, heating elements can be provided as part of the orbital shake bioreactor system.

The term “pilot scale orbital shake bioreactor system” or “pilot scale orbital shake bioreactor” refers to a system analogous to the large scale orbital shake bioreactor system with the difference of handling bags with a nominal volume of about 200 liters and a working volume of at least 50 liters.

For purpose of this invention, the term “large scale orbital shaker” or “production scale orbital shaker” refers to a machine capable of performing orbital shaking at the large/production scale. In the preferred embodiment of the present invention, a large scale orbital shaker was specifically designed and manufactured by Adolf Kühner AG, Birsfelden, Switzerland to hold containment vessels of around 1,500 L. The agitation diameter was set at 100 mm. The shaker was equipped with the direct drive technology, which means: (1) The speed of the motor is the same as the speed of the container; (2) there is no mechanical power transfer e.g., by friction wheels, no belts that can break down and no other mechanical wear and tear—leading to little noise and low power consumption due to less mechanical friction. In addition there is no false reading of shaker speed because of slipping belts. Finally, the parallelogram ensured that the shaking movement on the tray is absolutely equal and orbital, independent of the distribution of the load.

For purpose of this invention, the term “containment vessel” refers to a structure or vessel or container, which can be mounted on an orbital shaker and in which the disposable bag can be placed, where the liquid containing the cells usually is inside the disposable bag. Containment vessels can have different shapes and volumes. They can be made out of different materials ranging from plastic to metal. In the preferred embodiment of the present invention, disposable bags were designed to fit into a round open 1,500 L containment vessel made of LLDPE (linear low density polethylene) with a diameter of 1,300 mm, a height of 1,250 mm and an 8 mm wall thickness (Plastomatic AG, Muttenz, Switzerland). A metallic conical shape was constructed to fit in the bottom of the open containment vessel, resulting in a height difference of 180 mm between the center and the containment vessel wall. In some instances, “container” is used as a synonym for “containment vessel”.

For purpose of this invention—unless clearly stated otherwise—the term “bag” refers to a disposable bag used to cultivate the cells within. Disposable bags are widely used in the biotechnology industry mainly for the purpose of sterile liquid handling. They are gamma sterilized and validated to match GMP requirements. More recently, and due to improvements made in material properties, disposable bags were designed for cell cultivation in Wave bioreactors or in stirred tank reactors with single-use contact parts. Such bags are equipped with sterile filters, connections and sampling ports. Normally, disposable bags are made with a polymeric film with at least three layers. The structural layer determines the overall mechanical behavior of the film. Then, a barrier layer defines the structure's permeability. Finally, the fluid contact layer combines inertness and good sealing properties. To match the regulatory requirements, the validation procedures for a new film consist in testing a variety of material properties, including tensile properties, flex durability, permeability and possible interactions with the fluid. Further, to monitor the pH and the dissolved oxygen, innovative optical sensors can be integrated in the disposable cell culture bags. Sensor spots are immobilized on the inner layer of the bag in contact with the fluid. Using optical methods, the sensors can be assessed from the outside through the polymeric film. Optical sensors avoid contamination risks and can be discarded together with the cell culture bag. Response time and long term stability of optical sensors were improved to match process requirements.

For purpose of this invention, the term “nominal volume” of a bag refers to the maximum volume of liquid a bag can be filled with. “Working volume” refers to the actual liquid volume within a bag. For example, a bag could have a nominal volume of 1,500 liters, i.e., the bag can hold a maximum of 1,500 liters of liquid, but the working volume can be 750 liters, i.e., only 750 liters of liquid are inside the bag.

For purpose of this invention, the term “production scale” means a bioreactor system with a nominal volume exceeding 1,000 liters and/or a working volume exceeding 600 liters. “Production scale” is used synonymously with “large scale” in the context of the present invention—unless clearly stated otherwise.

For purpose of this invention, the term “pilot scale” means a bioreactor system with a nominal volume of 200 liters.

For purpose of this invention, the term “at least” should mean equal or larger. For example, “at least 200 liters” should mean “200 liters or more than 200 liters”.

For purpose of this invention, the term “protein” means a polypeptide (native [i.e., naturally-occurring] or mutant), oligopeptide, peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic including peptoids with nitrogen based backbone. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

For purposes of this invention, “amino acid” refers to a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

For purposes of this invention, by the term “transgene” is meant a nucleic acid composition made out of DNA, which encodes a peptide, oligopeptide or protein. The transgene may be operatively linked to regulatory control elements in a manner which permits transgene transcription, translation and/or ultimately directs expression of a product encoded by the expression cassette in the producer cell, e.g., the transgene is placed into operative association with a promoter and enhancer elements, as well as other regulatory control elements, such as introns or polyA sequences, useful for its regulation. The composite association of the transgene with its regulatory sequences (regulatory control elements) is referred to herein as a “minicassette”, “expression cassette”, “transgene expression cassette”, or “minigene”. The exact composition of the expression cassette will depend upon the use to which the resulting (mini)gene transfer vector will be put and is known to the artisan (Sambrook 1989, Lodish et al. 2000). When taken up by a target cell, the expression cassette as part of the recombinant vector genome may remain present in the cell as a functioning extrachromosomal molecule, or it may integrate into the cell's chromosomal DNA, depending on the kind of transfer vector used. Generally, a minigene may have a size in the range of several hundred base pairs up to about 30 kb.

For purposes of this invention, the term “cell” means any prokaryotic or eukaryotic cell, either ex vivo, in vitro or in vivo, either separate (in suspension) or as part of a higher structure such as but not limited to organs or tissues.

For purposes of this invention, the term “host cell” means a cell that can be transduced and/or transfected by an appropriate gene transfer vector. The nature of the host cell may vary from gene transfer vector to gene transfer vector.

For purposes of this invention, the term “producer cell” means a cell that is capable of producing a recombinant protein or protein of interest. The producer cell itself may be selected from any mammalian cell. Particularly desirable producer cells are selected from among any mammalian species, including, without limitation, cells such as HEK 293, A549, WEHI, 3T3, 10T1/2, BHK, MDCK, COS 1, COS 7, BSC 1, BSC 40, BMT 10, VERO, WI38, HeLa, Saos, C2C12, L cells, HT1080, HepG2, CHO, NS0, Per.C6. The selection of the mammalian species providing the cells is not a limitation of this invention; nor is the type of mammalian cell, i.e., fibroblast, hepatocyte, tumor cell, etc. Frequently used producer cells or HEK 293 cells, BHK cells, NS0 cells, Per.C6 cells and CHO cells. Preferentially, a producer cell should be free of potential adventitious viruses.

For purposes of this invention, “transfection” is used to refer to the uptake of nucleic acid compositions by a cell. A cell has been “transfected” when an exogenous nucleic acid composition has crossed the cell membrane. A number of transfection techniques are generally known in the art. Such techniques can be used to introduce one or more nucleic acid compositions, such as a plasmid vector and other nucleic acid molecules, into suitable host cells. Frequently, cells are transfected with 25-kd linear polyethyleneimine. Other alternatives are transfection by means of electroporation, liposomes, dendrimers, or calcium phosphate.

For purposes of this invention, by “vector”, “transfer vector”, “gene transfer vector” or “nucleic acid composition transfer vector” is meant any element, such as a plasmid, phage, transposon, cosmid, chromosome, virus, virus capsid, virion, etc., which is capable of transferring and/or transporting a nucleic acid composition to a host cell, into a host cell and/or to a specific location and/or compartment within a host cell. Thus, the term includes cloning and expression vehicles, as well as viral and non-viral vectors and potentially naked or complexed DNA. However, the term does not include cells that produce gene transfer vectors such as retroviral packaging cell lines.

For purpose of this invention, the term “specific productivity” refers to the amount of the protein of interest that is produced by a single cell per day. For example a specific productivity of 20 pg/cell/day refers to the production of 20 pg of the protein of interest by a single cell within 24 hours.

For purpose of this invention, the term “batch” refers to the (specific lot of) protein molecules of interest produced in a single production run, i.e., under the same production conditions. Batch means a specific quantity of a drug or other material that is intended to have uniform character and quality, within specified limits, and is produced according to a single manufacturing order during the same cycle of manufacture.

For purpose of this invention, the term “lot” means a batch, or a specific identified portion of a batch, having uniform character and quality within specified limits; or, in the case of a drug product produced by continuous process, it is a specific identified amount produced in a unit of time or quantity in a manner that assures its having uniform character and quality within specified limits

For purpose of this invention, the term “batch yield” refers to the maximum amount (in grams) of the recombinant protein of interest produced by all of the mammalian cells in the culture batch together. For secreted proteins, the “batch yield” refers to the maximum amount of the recombinant protein of interest in the culture medium where the recombinant protein of interest is secreted into the medium by the mammalian cells present in the medium. For example, if a mammalian cell culture of 1 liter comprises 0.5 g of recombinant protein of interest in total, the batch yield is 500 mg and the batch titer is 500 mg/l. Thus, whereas the specific productivity refers to the production of recombinant protein by a single mammalian cell within one day, the batch yield refers to the maximum amount of recombinant protein produced by all the mammalian cells in the culture during the total time of the culture. “Volumetric yield” can be used as a synonym for “batch yield”.

For purpose of this invention, the term “batch titer” refers to the maximum concentration (in grams per liter or milligrams per liter) of the recombinant protein of interest produced by all of the mammalian cells in the culture batch together. For secreted proteins, the “batch titer” refers to the maximum concentration of the recombinant protein of interest in the culture medium where the recombinant protein of interest is secreted into the medium by the mammalian cells present in the medium. For example, if a mammalian cell culture of 1 liter comprises 0.5 g of recombinant protein of interest in total, the batch yield is 0.5 grams and the batch titer is 0.5 g/l. Thus, whereas the specific productivity refers to the production of recombinant protein by a single mammalian cell within one day, the batch titer refers to the maximum concentration of recombinant protein produced by all the mammalian cells in the culture during the total time of the culture. The batch titer could also be defined as batch yield divided by culture volume.

For purpose of this invention, “growth medium” refers to a cell culture medium that promotes cell growth and division—leading to an increase in biomass as it relates to the cells. Optimally, a growth medium allows for a fast increase in biomass and supports cell growth to high cell densities.

For purpose of this invention, “transfection medium” refers to a cell culture medium that is suitable for transfection. Transfection media do not necessarily support cell growth or production. For example, RPMI can be used as transfection medium, but is not well suited for cell growth or production. An optimal transfection medium does not interfere with the transfection process, e.g., it does not contain inhibitors that inactivate the transfection reagent.

For purpose of this invention, “production medium” refers to a cell culture medium that promotes production of the protein of interest. A production medium does not necessarily support cell growth. Furthermore, one cannot necessarily transfect in production media, or only at a low transfection efficacy. An optimal production medium has the following characteristics: It sustains cell viability at a high cell density and results in high specific productivity for an extended period of time.

### (2) General Methods

The practice of the present invention will employ, unless otherwise indicated, conventional methods of microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

### (3) Preferred Embodiment, i.e., Best Mode Contemplated by the Inventors of Carrying Out the Present Invention

In the preferred embodiment, the cells used were CHO cells. These CHO cells produced a recombinant monoclonal antibody (anti-Rhesus D antibody, “CHO AMW cells”).

In the preferred embodiment bags containing more than 600 liter of working volume and more than 1,000 liter nominal volume were tested and found to provide sufficient oxygen transfer and mixing for typical cell cultures. The preferred embodiment describes [A] the large scale orbital shake bioreactor system, [B] the cell expansion phase, [C] the production phase, and [D] the results obtained.

[A] Large Scale Orbital Shake Bioreactor System

The large scale orbital shake bioreactor system—capable of handling bags exceeding 600 liter working volume and exceeding 1,000 liter nominal volume in general, and 750 liter working volume and 1,500 liter nominal volume in particular—comprises at least the following components: (1) An orbital shaker, (2) a containment vessel (to place a disposable plastic bag inside), which is mounted on the orbital shaker, and (3) a disposable plastic bag, which contains the cells in a liquid medium. The system might further comprise two optional components, a heating system (4) and optical sensors (5). As for temperature control, the orbital shake bioreactor can be placed in a temperature-controlled environment, which is not part of the orbital shake bioreactor (e.g., a 37° C. room), which eliminates the need for an endogenous heating system. In the preferred embodiment, the heating system was an endogenous part of the orbital shake bioreactor system.

In the preferred embodiment, the nominal volume of the disposable plastic bag was 1,500 liters, the working volume was 750 liters.

The large scale orbital shake bioreactor system was designed with the following main features:
Efficient orbital shake technology
Convenient disposable cell culture bags
Cost-effective
Low energy consumption
Maintenance free
Non-invasive optical monitoring of pH and DO (DO: dissolved oxygen).

Orbital Shaker (1)

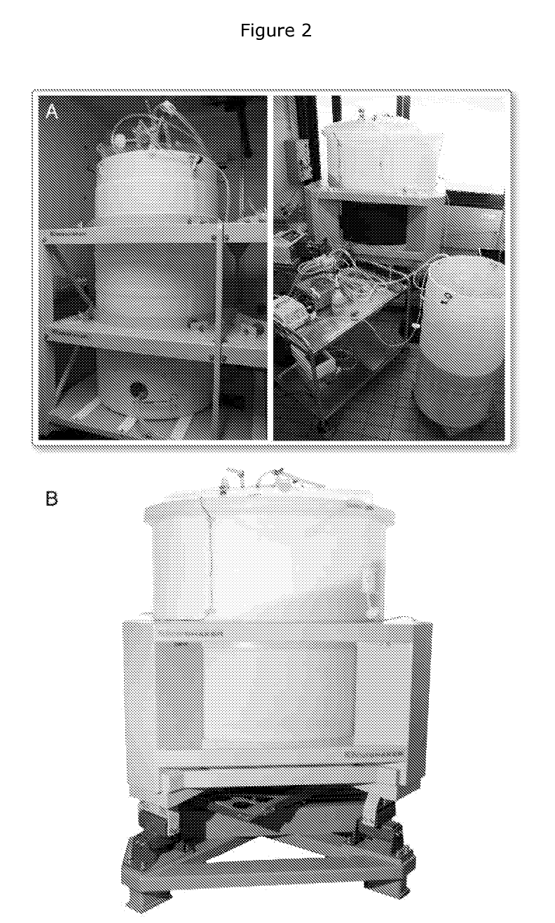

A large scale orbital shaker was specifically designed and manufactured by Adolf Kühner AG, Birsfelden, Switzerland to hold containment vessels up to around 1,500 L. The agitation diameter was set at 100 mm. The shaker was equipped with the direct drive technology, which means: (1) The speed of the motor is the same as the speed of the container; (2) there is no mechanical power transfer, e.g., by friction wheels, no belts that can break down and no other mechanical wear and tear—leading to little noise and low power consumption due to less mechanical friction. In addition there is no false reading of shaker speed because of slipping belts. Finally, the parallelogram ensured that the shaking movement on the tray was absolutely equal and orbital, independent of the distribution of the load (FIG. 2 in general and FIG. 2B in particular).

Single-Use Cell Culture Bags (3)

Disposable bags are widely used in the biotechnology industry mainly for the purpose of sterile liquid handling. Plastic film technologies improved in terms of mechanical resistance and other desirable material properties. They are gamma sterilized and validated to match GMP requirements.

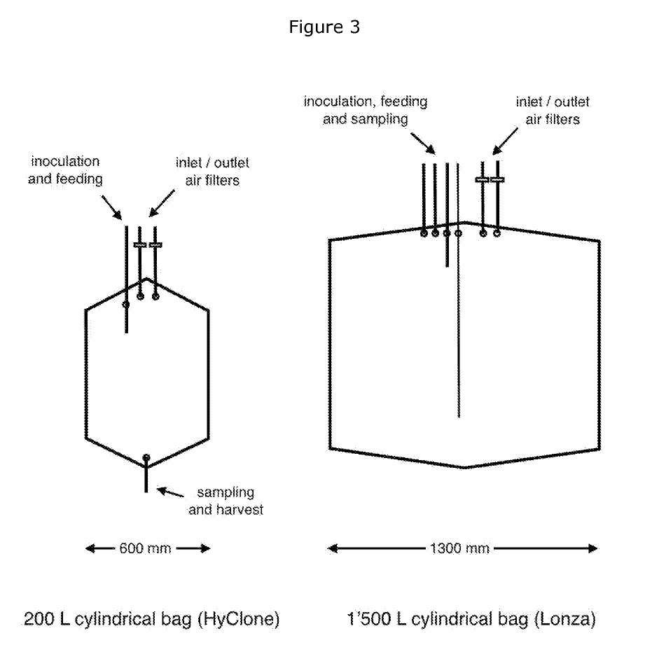

Since no appropriate standard bag was available for production scale tests, a cylindrical, 1,500 L cell culture bag was designed in collaboration with Lonza (Lonza SPRL, Verviers, Belgium). The sterile bags were equipped with ports and connections located on the top for inoculation, feeding, and sampling (FIG. 3). Inlet and outlet sterile air filters were connected to ports located on the top. The bags were designed to fit into a round open 1,500 L containment vessel.

Containment Vessel (2)

The disposable bags were designed to fit into a round open 1,500 L containment vessel made of LLDPE (linear low density polethylene) with a diameter of 1,300 mm, a height of 1,250 mm and a 8 mm wall thickness (Plastomatic AG, Muttenz, Switzerland). A metallic conical shape was constructed to fit in the bottom of the open container, resulting in a height difference of 180 mm between the center and the container wall.

Heating System (4)

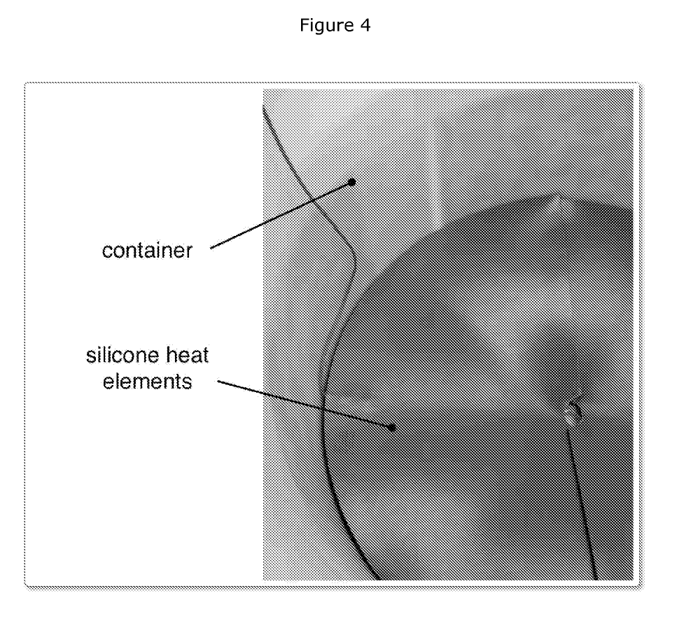

Silicone heat elements (Prang+Partner AG, Pfungen, Switzerland) were used to maintain the temperature of the cell culture suspension in the production scale orbital shake bioreactor (1,500 L bag). Large half-circle silicon heaters were adjusted to the container conical bottom (FIG. 4). The cell culture bag was placed in direct contact with the heating elements. A 10 mm thick neoprene insulation sheet was used to insulate the container wall from the outside environment. A PT-100 temperature probe was inserted between the container inner wall and the cell culture bag at a height of 200 to 300 mm from the bottom. A thermostatic temperature controller was used to maintain the temperature of the well-mixed bioreactor at ±0.5° C. of the set point. The temperature operating range was 22° C. (room temperature) to 45° C.

Optical System for Sensing of pH or Dissolved Oxygen (5)

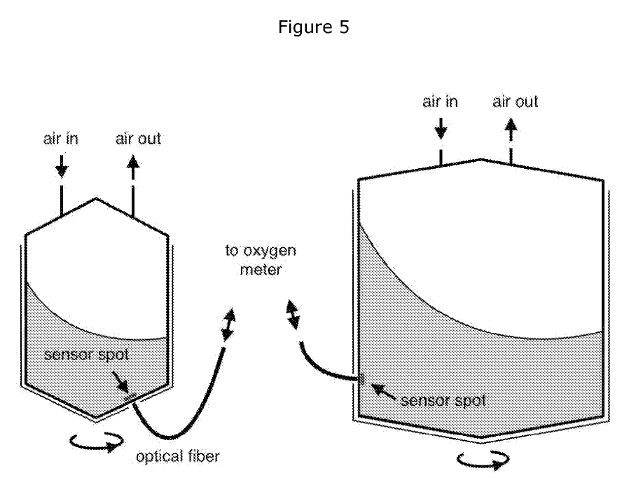

For oxygen transfer evaluations in the large scale orbital shake bioreactor, an optical sensing set-up was used. Oxygen sensor spots were fixed with silicone glue on the inside layer of the plastic film of the disposable bag. In the 1,500 L bag, the spot was placed on the lateral inner wall at a height of 300 mm from the bottom (FIG. 5). Small openings were created in the containment vessels to place the optical fiber in contact with the outer layer of the plastic film of the disposable bag.

A normal electrochemical dissolved oxygen electrode was mounted on the closure. Use of optical sensors for pH and dissolved oxygen (PreSens GmbH, Germany; www.presens.de/htmL/start.htmL) left more space on the closure.

[B] Cell Expansion Phase

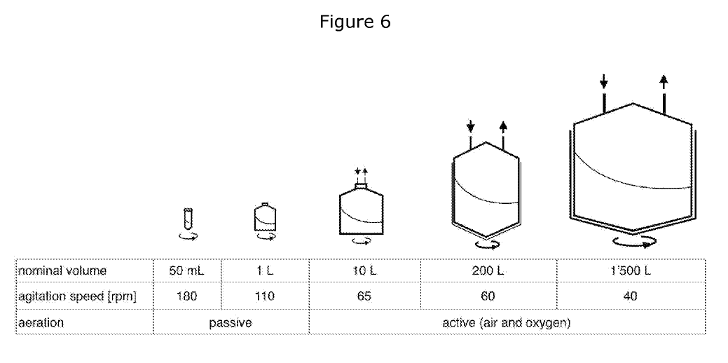

As mentioned, the cells used in the preferred embodiment were CHO cells producing a recombinant monoclonal antibody (anti-Rhesus D; CHO AMW cells). To expand the cells for the production phase in large scale operations, shake bioreactor systems of increasing volumes were successively used. The scale-up sequence comprised shake bioreactors of the following nominal volumes: 50 mL, 1 L, 10 L and 200 L (FIG. 6).

The latter one (“pilot scale 200 L orbital shake bioreactor”) was used to inoculate the production/large scale shake bioreactor. Each of these systems reached cell densities between 4 and 6×10⁶ cells mL⁻¹. Lab-scale systems (50 mL and 1 L) were passively aerated. The caps were fitted with a sterile hydrophobic membrane for passive gas diffusion from the environment into the vessel headspace.

At pilot and production scale, the airflow rate through the headspace was actively controlled using a membrane pump. The pH was manually adjusted by varying the CO₂ concentration in the inlet airflow.

The 1,500 L production scale orbital shake bioreactor system was supplied with the desired volume of cell culture medium using sterile connections and a sterile filtration step (0.22 μm). The medium was heated up overnight to 37° C. at a low to moderate shaking speed. (In stirred tank bioreactors, the use of steam and heating jackets results in brief heat-up times. However, similar heat exchange systems were inappropriate for large-scale disposable shake bioreactors, and heating times are significantly longer. For a liquid volume of 750 L, heat-up times of 10-12 h resulted. Tests with larger heat element contact surfaces and improved insulation might result in heat-up times of a few hours only.) Then, the 200 L scale-up bioreactor was connected to the 1,500 L scale system for inoculation.

To summarize the last steps of the expansion phase: Prior to inoculating the production scale orbital shake bioreactor, cell expansion was accomplished in the 200 L pilot scale shake bioreactor with a working volume of 75 L. A relatively inexpensive serum- and protein-free medium was used for this purpose (CHO PFM). As expected, slower growth kinetics resulted with a maximal cell density of 4×10⁶ cells mL⁻¹. For the production scale, serum-free ProCHO5 medium was used. As shown at smaller scales, this medium usually supports growth of CHO cells up to 6-8×10⁶ cells mL⁻¹.

First, 500 L medium were transferred into the 1,500 L cell culture bag using a sterile filtration step. The next day, when the temperature reached 37° C., the 1,500 L shake bioreactor was inoculated at a density of 4×10⁵ cells mL⁻¹. Then, medium was added to reach a final working volume of 750 L.

[C] Production Phase

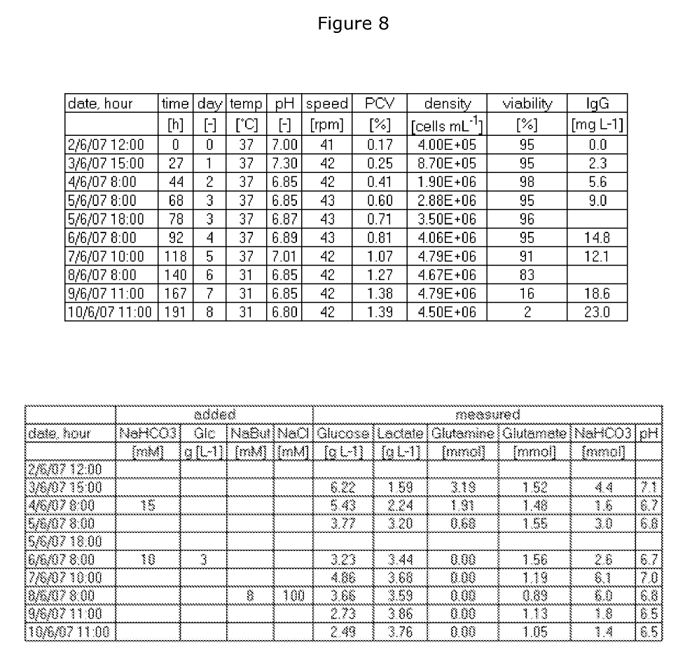

The disposable bag within the containment vessel of the production scale orbital shake bioreactor was filled with the desired volume of cell culture medium using sterile connections and a sterile filtration step as outlined in [B] and FIG. 2. With a final cell culture volume of 750 L, an agitation speed of 40 to 45 rpm was applied, depending on cell density (for details of the operation parameters see also FIG. 8). During the exponential growth phase, 3 g L⁻¹ glucose and 25 mM NaHCO₃ were fed to sustain the growth and maintain a physiological pH. An airflow rate of 10-20 L min⁻¹ was provided. At a cell density of 3×10⁶ cells mL⁻¹, pure oxygen was used instead of air at a lower flow rate (5-10 L min⁻¹). The outlet air filter was heated up to avoid condensation. Samples were taken daily for the monitoring of cell density, viability, packed cell volume, pH and recombinant protein production of a monoclonal antibody. Glucose and sodium bicarbonate levels were measured and adjusted by feedings. On day 5, a maximal total cell density of approximately 4.8×10⁶ cells mL⁻¹ was assessed with a viability of 91%.

[D] Results

FIG. 8 summarizes some key results and parameters of the preferred embodiment in an overview table.

Result 1: Growth Kinetics in 200 and 1,500 L Orbital Shake Bioreactors

With the set-up described above, the production-scale orbital shake bioreactor allowed reliable cell growth with up to 750 L cell culture volume. Unlike stirred tank bioreactors, where the energy input is due to the impeller, for shake cultivation systems, the wetted contact area between the rotating liquid and the vessel is regarded as the “stirring element”.

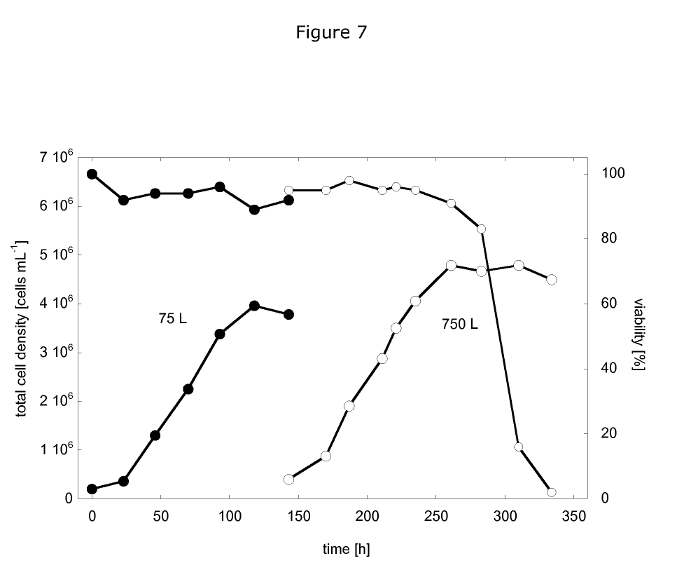

FIG. 7 shows total cell density and viability for the 200 L and 1,500 L orbital shake bioreactors. As one can see, on day 5 (˜120 h), a maximal total cell density of 4.8×10⁶ cells mL⁻¹ was assessed with a viability of 91% in the 1,500 L orbital shake bioreactor. These results confirm the assumptions made previously that orbital shake technology is particularly well-suited for growing mammalian cells, even at the production scale. The use of non-invasive optical sensors facilitated the monitoring and control of the dissolved oxygen and the pH.

Result 2: Antibody Titers Achieved in the 1,500 L Orbital Shake Bioreactor System

The following antibody titers were obtained by cultivating CHO AMW cells (De Jesus et al. 2004), which produce a monoclonal anti-Rhesus D antibody, in the 1,500 L orbital shake bioreactor system according to the teachings of the preferred embodiment:
0 hours (after inoculation): 0 mg/l (antibody titer)
27 hours (after inoculation): 2.3 mg/l (antibody titer)
44 hours (after inoculation): 5.6 mg/l (antibody titer)
68 hours (after inoculation): 9.0 mg/l (antibody titer)
92 hours (after inoculation): 14.8 mg/l (antibody titer)
118 hours (after inoculation): 12.1 mg/l (antibody titer)
167 hours (after inoculation): 18.6 mg/l (antibody titer)
191 hours (after inoculation): 23.0 mg/l (antibody titer)

Antibody titers were determined by ELISA as described by Meissner et al. (2001). In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™ 340; Molecular Devices, Palo Alto, Calif., USA).

1. A method for the cultivation of cells in a bioreactor system comprising the not necessarily consecutive steps of:
(1) providing a bag having a nominal volume exceeding 1,000 liters;
(2) placing said bag into a containment vessel;
(3) securing said containment vessel with the bag onto a platform of an orbital shaker;
(4) introducing a liquid medium and cells into the bag, so that the working volume exceeds 600 liters;
(5) filling the remainder of the maximum bag volume with a gas;
(6) shaking the platform to thereby induce motion to the liquid medium in the bag, whereby the necessary oxygen transfer and mixing required for cell growth and/or survival is accomplished by the motion of the shake mixing.

2. A method for the production of a recombinant protein in a bioreactor system comprising the not necessarily consecutive steps of:
(1) providing a bag having a nominal volume exceeding 1,000 liters;
(2) placing said bag into a containment vessel;
(3) securing said containment vessel with the bag onto a platform of an orbital shaker;
(4) introducing a liquid medium and cells into the bag, so that the working volume exceeds 600 liters;
(5) filling the remainder of the maximum bag volume with a gas;
(6) shaking the platform to thereby induce motion to the liquid medium in the bag, whereby the necessary oxygen transfer and mixing required for cell survival and/or productivity is accomplished by the motion of the shake mixing.

3. A method for the cultivation of cells in a bioreactor system comprising the not necessarily consecutive steps of:
(1) providing a bag having a nominal volume of 1,500 liters;
(2) placing said bag into a containment vessel;
(3) securing said containment vessel with the bag onto a platform of an orbital shaker;
(4) introducing a liquid medium and cells into the bag, wherein the liquid medium and the cells comprise 50% of the nominal volume and thus define the working volume of 750 liters;
(5) filling the remainder of the maximum bag volume with a gas;
(6) orbitally shaking the platform to thereby induce motion to the liquid medium in the bag, whereby the necessary oxygen transfer and mixing required for cell growth and/or survival is accomplished by the motion of the shake mixing

4. A method for the production of a recombinant protein in a bioreactor system comprising the not necessarily consecutive steps of:
(1) providing a bag having a nominal volume of 1,500 liters;
(2) placing said bag into a containment vessel;
(3) securing said containment vessel with the bag onto a platform of an orbital shaker;
(4) introducing a liquid medium and cells into the bag, wherein the liquid medium and the cells comprise 50% of the nominal volume and thus define the working volume of 750 liters;
(5) filling the remainder of the maximum bag volume with a gas;
(6) orbitally shaking the platform to thereby induce motion to the liquid medium in the bag, whereby the necessary oxygen transfer and mixing required for cell survival and/or productivity is accomplished by the motion of the shake mixing.

5. The method of claims 1, 2, 3 or 4, wherein the orbital shaking takes place at an agitation speed between 30 rpm and 45 rpm.

6. The method of claims 1, 2, 3 or 4, wherein the orbital shaking takes place at an agitation speed between 40 rpm and 45 rpm.

7. The method of claims 1, 2, 3 or 4, wherein the agitation diameter of the bioreactor can be varied from 50 mm to 150 mm.

8. The method of claims 1, 2, 3 or 4, wherein the agitation diameter of the bioreactor is 100 mm.

9. The method of claims 1, 2, 3 or 4, further comprising the steps of: introducing gas containing oxygen and/or carbon dioxide into the bag during the orbital shaking step; and exhausting products of respiration from the bag during the orbital shaking step.

10. The method of claims 1, 2, 3 or 4, further comprising the steps of: introducing gas containing oxygen and/or carbon dioxide into the bag during the orbital shaking step; and exhausting products of respiration from the bag during the orbital shaking step, wherein the steps of introducing the gas and exhausting the products of respiration during the orbital shaking step further comprise introducing the gas and exhausting the products of respiration at a controlled rate.

11. The method of claims 1, 2, 3 or 4, wherein said cells are of animal, human, insect, microbial, or plant origin.

12. The method of claims 1, 2, 3 or 4, wherein said cells are used for the production of a protein.

13. The method of claims 1, 2, 3 or 4, wherein said cells are CHO cells.

14. The method of claims 1, 2, 3 or 4, wherein said cells are HEK293 cells.

15. The method of claim 2 or 4, wherein said protein is an immunoglobulin.

16. The method of claims 1, 2, 3 or 4, wherein said cells are cultivated for the purpose of biomass expansion.

17. The method of claims 1, 2, 3 or 4, wherein said cells are cultivated for the purpose of protein production.

18. The method of claims 1, 2, 3 or 4, wherein said cells are used for the production of a virus.

19. The method of claims 1, 2, 3 or 4, wherein said bag is inflated.

20. The method of claims 1, 2, 3 or 4, wherein said bag is disposable.

21. The method of claims 1, 2, 3 or 4, wherein said bag is a plastic bag.

22. The method of claims 1, 2, 3 or 4, wherein said bag is a plastic bag made out of polypropylene, polycarbonate or polyethylene.

23. The method of claims 1, 2, 3 or 4, said bag is sterilized.

24. The method of claims 1, 2, 3 or 4, wherein said bag is cylindrical.

25. The method of claims 1, 2, 3 or 4, wherein said bag is equipped with ports and connections located on the top for inoculation, feeding or sampling.

26. The method of claims 1, 2, 3 or 4, wherein said containment vessel is a plastic vessel.

27. The method of claims 1, 2, 3 or 4, wherein said containment vessel is made out of polypropylene, polycarbonate, polyethylene or LLDPE (linear low density polyethylene).

28. The method of claims 1, 2, 3 or 4, wherein said containment vessel is a metal vessel.

29. The method of claims 1, 2, 3 or 4, wherein said containment vessel is cylindrical, square shaped, conical or spherical.

30. The method of claims 1, 2, 3 or 4, wherein said containment vessel is cylindrical.

31. The method of claims 1, 2, 3 or 4, wherein said containment vessel has a diameter of 1.3 m, a height of 1.25 m and a 8 mm wall thickness.

32. The method of claims 1, 2, 3 or 4, wherein a shape is placed at the bottom of the containment vessel, resulting in a height difference of 100 mm to 300 mm between the center and the containment vessel wall.

33. The method of claims 1, 2, 3 or 4, wherein shape is placed at the bottom of the containment vessel, resulting in a height difference of 100 mm to 300 mm between the center and the containment vessel wall, and said shape is a metallic conical shape.

34. The method of claims 1, 2, 3 or 4, wherein shape is placed at the bottom of the containment vessel, resulting in a height difference of 180 mm between the center and the containment vessel wall, and said shape is a metallic conical shape.

35. The method of claims 1, 2, 3 or 4, wherein said bioreactor system is a non-instrumented bioreactor system.

36. The method of claims 1, 2, 3 or 4, wherein said bioreactor system is not actively provided with oxygen and/or carbon dioxide.

37. The method of claims 1, 2, 3 or 4, wherein said bioreactor system further comprises an optical sensor which is used to measure the pH or dissolved oxygen within the bioreactor system.

38. The method of claims 1, 2, 3 or 4, wherein said bioreactor system is equipped with the direct drive technology, which means that the speed of the motor is the same as the speed of the container.

39. The method of claims 1, 2, 3 or 4, wherein a parallelogram mechanism ensures that the shaking movement on the platform is equal and orbital, independent of the load distribution.

40. The method of claims 1, 2, 3 or 4, wherein the cells in suspension are maintained at 37° C.

41. The method of claims 1, 2, 3 or 4, further comprising a heating system.

42. The method of claims 1, 2, 3 or 4, further comprising a silicone heating system where large half-circle silicon heaters are adjusted to the containment vessel conical bottom, the cell culture bag is placed in direct contact with the heating elements, and a 10 mm thick neoprene insulation sheet is used to insulate the containment vessel wall from the outside.

43. The method of claims 1, 2, 3 or 4, further comprising a PT-100 temperature probe which is inserted between the containment vessel inner wall and the cell culture bag at a height of 200 mm from the bottom.

44. The method of claims 1, 2, 3 or 4, further comprising a thermostatic temperature controller, which allows maintaining the temperature of the medium at ±0.5° C. of a set point.

45. The method of claims 1, 2, 3 or 4, wherein said bag is placed into the containment vessel after introducing a liquid media with or without cells into the bag.

46. The method of claims 1, 2, 3 or 4, wherein said containment vessel is secured onto the platform prior to placing said bag into the containment vessel.

47. The method of claims 1, 2, 3 or 4, wherein said containment vessel with the bag is secured onto the platform of the orbital shaker after introducing a liquid media and cells into the bag.

48. The method of claims 1, 2, 3 or 4, wherein liquid media with or without cells is introduced in said bag, prior and/or after placing said bag into a containment vessel and prior and/or after securing said containment vessel onto the platform of the orbital shaker.
